(19) United States
(12) Patent Application Publication
(10) Pub. No.: US 2023/0219015 A1
(43) Pub. Date: Jul. 13, 2023

(54) EXTRACTION METHOD AND MIXTURE OF SUBSTANCES

(71) Applicant: K.D. PHARMA BEXBACH GMBH, Bexbach (DE)

(72) Inventors: Gideon GIESSELMANN, Tawern (DE); Roberto FRONZONI, Homburg (DE); Denitsa SHOPOVA-GOSPODINOVA, Schönenberg-Kübelberg (DE); Elia RAMOS-TERCERO, Saarbrücken (DE)

(21) Appl. No.: 18/022,869

(22) PCT Filed: Aug. 26, 2021
(86) PCT No.: PCT/EP2021/073585

§ 371 Date: Feb. 23, 2023

(30) Foreign Application Priority Data
Aug. 26, 2020 (LU) LU102018
Jul. 22, 2021 (LU) LU500463

(51) Int. Cl. B01D 11/02; C07C 37/72; C07C 51/48; C07D 311/80; C07B 63/00

(52) U.S. Cl. CPC B01D 11/0288; C07C 37/72; C07C 51/48; C07D 311/80; C07B 63/00; C07C 2601/16

(57) ABSTRACT

A method for extraction, in which an extraction agent is used for dissolving at least one extraction material out of a substance. The extraction agent includes fatty acid ethyl ester and/or fatty acid methyl ester. In one embodiment the extraction agent is an omega-3 and/or omega-6 fatty acid, preferably EPA, DHA, DPA, ETA, 21:5n3 and/or SDA. In one embodiment the extraction agent includes at least 500 mg/g, preferably at least 700 mg/g of the fatty acid ethyl ester and/or of the fatty acid methyl ester. Expediently, the mixture of substances is a plant material, preferably hemp, and the extraction material is a cannabinoid, preferably CBD and/or THC.

Extraction Method and Mixture of Substances

The invention relates to a method for extraction in which an extractant is used to dissolve at least one extraction material out of a substance, in particular a plant material.

The invention further relates to a substance mixture containing an extraction material extracted from a substance, in particular a plant material.

Through use, various extraction methods are known in which different extractants are used. Ethanol or supercritical CO₂ is usually used for extraction from plant material. In particular, the use of these extractants has also proven to be suitable for extracting cannabinoids such as THC or CBD from hemp. Disadvantages of these methods are that they are energy-intensive and unwanted byproducts are found in the enriched products.

Methods for extracting cannabinoids from hemp are known from US 2016/0213720 A1 and WO 2018/130682 A1.

The invention is based on the object of providing a method of the type mentioned at the outset that enables a more efficient extraction in comparison with the known methods.

This object is achieved according to the invention in that the extractant comprises fatty acid ethyl ester or/and fatty acid methyl ester.

It has surprisingly been found that the extraction material can be removed from the substance particularly well when using fatty acid ethyl ester or/and fatty acid methyl ester, especially if the substance is a plant. The method has proven to be particularly advantageous for the extraction of cannabinoids, in particular phytocannabinoids. The extraction is preferably effected from hemp. Not only is a comparatively large extraction yield achieved, but the method can also be carried out efficiently in a comparatively large temperature range. The invention proves to be particularly advantageous for the extraction of medical hemp, for example of the Cannabis sativa L. ‘Afina’ variety, the Cannabis sativa L. ‘Ludina’ variety, the Cannabis sativa L. ‘Elida’ variety, the Cannabis sativa L. ‘Talea’ variety and/or the Cannabis sativa L. ‘Rensina’ variety.

In addition to the application to hemp, the method can advantageously also be used for the extraction of extraction material from other plants or constituents thereof, for example
α-caryophyllene and/or β-caryophyllene from hops,
a and/or B acids from hops,
sesquiterpenes from Valerianoideae,
ginkgolide terpene lactones and/or flavone glycosides from Ginkgo biloba,
flavonoids from Erythroxylum coca,
linoleic acid and/or linolenic acid from poppy,
thymol and/or sesquiterpene lactones from arnica,
cineole and/or cymene from eucalyptus,
catechins and/or theanines from tea plants (e.g. Camellia sinensis, Camellia assamica),
termineol and/or pinene from rosemary,
hydroxycinnamic acid and/or citronellal from melissa,
gingerol and/or zingiberol from ginger,
thymol and/or carvacrol from thyme,
cinnamaldehyde eugenol from cinnamon,
linalool, α-pinene and/or γ-terpine from coriander,
menthol and/or menthone from mint,
linalyl acetate and/or coumarins from lavender,
thujanes and/or borneol from sage,
isoabienol, monoterpenes or/and carene from conifers.

Furthermore, the method is suitable for producing the substance mixture mentioned at the outset comprising fatty acid ethyl ester or/and fatty acid methyl ester and containing an extraction material extracted from a substance, in particular a plant material. It is conceivable to flavor fatty acid ethyl ester or/and fatty acid methyl ester by means of the method. Furthermore, it is possible to enrich fatty acid ethyl ester or/and fatty acid methyl ester with the extraction material by means of the method.

In one configuration of the invention, the fatty acid ethyl ester contained in the substance mixture or/and the fatty acid methyl ester forms the extractant.

In one embodiment of the invention, the substance mixture is treated after the extraction, optionally for purification, preferably by means of filtering, particularly preferably by means of an activated carbon filter. Such a treatment makes it possible to remove, for example, chlorophyll from the substance mixture.

The fatty acid ethyl ester or/and fatty acid methyl ester is usefully of pharmaceutical quality. It has in particular been produced under the conditions according to the rules of the “Good Manufacturing Practice (GMP) for Medicinal Products”, preferably in accordance with the Arzneimittel- und Wirkstoffherstellungsverordnung (AMWHV, German Ordinance on the Manufacture of Medicinal Products and Active Pharmaceutical Ingredients). It preferably corresponds to the purity specified in the United States Pharmacopeia-National Formulary, British Pharmacopoeia or European Pharmacopoeia.

In a particularly preferred embodiment of the invention, the fatty acid ethyl ester comprises or is an omega-3 and/or omega-6 fatty acid. Advantageously, an omega-3 and/or omega-6 fatty acid that is enriched with the extraction material may be formed by means of the method. Omega-3 and/or omega-6 fatty acid is known to have particularly health-promoting effects as a foodstuff and supplement.

The invention proves to be particularly advantageous it after the extraction, the extraction material is present, in particular dissolved, in the extractant, in particular the omega-3 and/or omega-6 fatty acid. The method may then be used to directly produce a fatty acid ethyl ester or/and fatty acid methyl ester enriched with the extraction material. By means of the method according to the invention, combination products can be produced directly and therefore in a comparatively simple manner. There is thus no need to first remove the extraction material from the substance, in particular the plant, by means of another method and to then admix the fatty acid ethyl ester, in particular the omega-3 and/or omega-6 fatty acid, with the extraction material.

The invention has proven to be particularly advantageous if the extractant comprises or is EPA, DHA, DPA, ETA, 21:5n3 or/and SDA. These omega-3 fatty acids, in particular EPA and DHA, are particularly healthy foodstuffs or supplements.

Fatty acid methyl ester is used in many applications as a vegetable fuel. In addition to the use according to the invention as the extractant, the invention provides the possibility of enriching fatty acid methyl ester with the extraction material in a particularly simple manner.

Advantages of using the fatty acid methyl ester as extractant are that it can be produced sustainably from vegetable fats and is cost-effective and readily available. Furthermore, the extractant can be used for short-path distillation in a particularly economically viable manner. The use of the fatty acid methyl ester has proven to be particularly advantageous when extracting from hemp. Cannabinoids, in particular phytocannabinoids, and terpenes also present in hemp can be separated using fatty acid methyl ester by way of short-path distillation. The separation may in particular be effected via different boiling points.

In one embodiment of the invention, the extractant is a substance mixture, preferably an oil.

In a further configuration of the invention, a further organic solvent is used as extractant in addition to the fatty acid ethyl ester or the fatty acid methyl ester.

While it would be conceivable, as the extractant, to mix the fatty acid ethyl ester and/or the fatty acid methyl ester with the organic solvent, in the method at least two of the different extractants mentioned, fatty acid ethyl ester or/and fatty acid methyl ester and the organic solvent, may be used for the extraction one after the other, optionally in separate extraction steps. The further organic solvent is usefully an alcohol, preferably ethanol, methanol, n-butanol and/or isopropanol.

In one embodiment of the invention, the extractant comprises at least 90 mg/g, preferably at least 120 mg/g, of fatty acid ethyl ester, preferably EPA and/or DHA, and/or at least 175 mg/g, preferably at least 190 mg/g, of fatty acid methyl ester.

However, it has proven to be particularly advantageous to use an extractant comprising at least 500 mg/g, preferably at least 700 mg/g, particularly preferably at least 900 mg/g, of fatty acid ethyl ester, preferably EPA and/or DHA; and/or at least 500 mg/g, preferably at least 700 mg/g, particularly preferably at least 900 mg/g, of fatty acid methyl ester. As a result, not only can a particularly great extraction effect be achieved, but it is optionally possible, using the fatty acid ethyl ester as extractant, to directly form a product intended for consumption, in particular a supplement, that is admixed with the extraction material.

In a particularly preferred embodiment of the invention, the extraction method is carried out such that the substance mixture contains the extraction material, in particular the cannabinoid, possibly the phytocannabinoid, preferably the CBD, the THC and/or the CBD-A, in a content of at least 5% by weight, preferably at least 25% by weight, particularly preferably 50% by weight. With a cannabinoid content of up to 10% by weight, the substance mixture is suitable as a foodstuff or supplement. With greater contents, in particular with a content of at least 25% by weight, preferably of at least 50% by weight, the substance mixture is suitable for medical use or as a medical product.

In a particularly preferred configuration of the invention, the component to be dissolved out is a cannabinoid, in particular a phytocannabinoid, preferably CBD and/or THC, and/or a terpene. The substance from which the extraction material is extracted is preferably hemp, particularly preferably medical hemp, in particular of the Cannabis sativa L. ‘Afina’ variety, the Cannabis sativa L. ‘Ludina’ variety, the Cannabis sativa L. ‘Elida’ variety, the Cannabis sativa L. ‘Talea’ variety and/or the Cannabis sativa L. ‘Rensina’ variety.

Omega-3 and/or omega-6 fatty acids are metabolized in the human body partly to form epoxides. These epoxides can be converted by cytochrome P450 into omega-3 and/or omega-6 fatty acid endocannabinoid epoxides. These endogenous endocannabinoid epoxides have proven to be particularly bioactive. They have an anti-inflammatory, vasodilatory (vessel-expanding) effect and can modulate platelet aggregation. In addition, the omega-3 and/or omega-6 fatty acids have proven to be advantageous for the effectiveness of the cannabinoids, in particular the phytocannabinoids. Omega-3 and/or omega-6 fatty acids act as anchors in the cell membrane for the endocannabinoids and for cannabinoids, in particular phytocannabinoids. They are involved in the synthesis of receptors of the endocannabinoid system, which forms part of the human or animal nervous system. The combination of cannabinoids, in particular phytocannabinoids, with omega-3 and/or omega-6 fatty acids can thus contribute to an improvement in the effects brought about by the two substances.

The use of omega-3 and/or omega-6 fatty acids enriched with cannabinoids, in particular phytocannabinoids, is considered to be particularly advantageous for use in pain therapy and/or chemotherapy, since they exhibit a synergistic effect when administered together. While the omega-3 and/or omega-6 fatty acids in particular have an anti-inflammatory effect and thereby already enable a reduction in analgesics on their own, the cannabinoids can in particular block the descending nerve pathways and thus permit a reduction in a necessary dose of analgesics.

The use of the combination according to the invention of omega-3 and/or omega-6 fatty acids and cannabinoids, in particular phytocannabinoids, is considered to be particularly advantageous when used alongside pain therapy with another analgesic, in particular an opioid, because the additional intake of the omega-3 and/or omega-6 fatty acid that is admixed with the cannabinoid, in particular phytocannabinoid, enables a particularly marked reduction in the necessary dose of the other analgesic, in particular the opioid.

It has proven to be advantageous to carry out the extraction at 15° C. to 45° C., preferably at 20° C. to 40° C.

The substance is usefully incubated before the extraction. If the substance is a plant material, it is preferably incubated between 80° C. and 120° C., preferably between 90° C. and 110° C.

In a refinement of the invention, the substance mixture mentioned at the outset containing an extraction material extracted from a substance, in particular a plant material, comprises fatty acid ethyl ester or/and fatty acid methyl ester.

In a particularly preferred configuration of the invention, the fatty acid ethyl ester and/or fatty acid methyl ester of the substance mixture is an extractant by means of which the extraction material has been extracted, the extraction material preferably being dissolved in the fatty acid ethyl ester and/or fatty acid methyl ester.

The substance mixture usefully contains the extraction material, in particular the cannabinoid, in particular the phytocannabinoid, preferably the CBD, the THC and/or the CBD-A, in a content of at least 3% by weight, preferably of at least 6% by weight, particularly preferably of at least 9% by weight.

In one embodiment of the invention, the substance mixture comprises substance components of the extraction material, in particular substance components of the cannabinoid, in particular phytocannabinoid, preferably CBD, THC and/or CBD-A, and/or of the terpene, in a ratio of the substance components to one another that corresponds, or at least substantially corresponds, to the substance component ratio of the extraction material, in particular the cannabinoid and/or terpene substance component ratio, in the plant material.

The invention is elucidated in more detail hereinafter on the basis of examples and the appended graphs which relate to the exemplary embodiments.

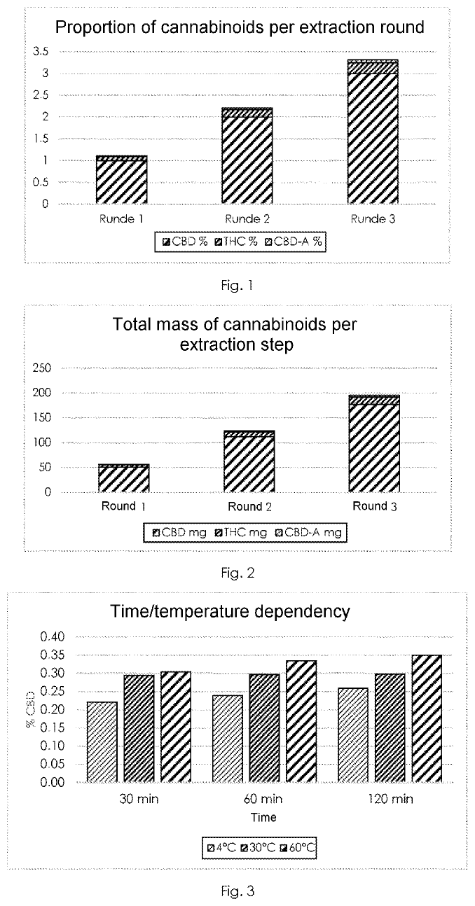

FIGS. 1 to 3 show substance contents after different methods or method steps have been carried out.

### 1^{ST} EXAMPLE

Hemp of the Finola variety was incubated for two hours at 100° C. in order to decarboxylate CBD-A into CBD and THC-A into THC.

10 g of the incubated hemp and 20 g of an omega-3 fatty acid oil that contained 96.5% EPA were then mixed and left to stand for 1 h at 23° C. Filtration was subsequently performed and the filtrate formed and the filter were washed with 30 g of ethanol in order to remove the omega-3 fatty acid oil from the filter and the filter cake. After evaporation of the ethanol, the weight (total g) was determined and the concentration of cannabinoids was measured.

For comparison purposes, 10 g of the incubated hemp was treated in the same way with 20 g of 100% ethanol.

After filtration, the contents of CBD and THC reported in the following table were determined:

TABLE 1

| | | CBD | THC | CBD-A |
|---|---|---|---|---|
| Finola [g] | 20 g | (% by weight) | (% by weight) | (% by weight) |
| 10 g | Ethanol | 0.5 | 0.04 | 0.06 |
| 10 g | EPA | 0.6 | 0.075 | 0.03 |

As can be seen from the results, both more CBD and more THC were able to be extracted when EPA was used. Furthermore, there was less CBD-A remaining when EPA was used.

### 2^{ND} EXAMPLE

Hemp of the Finola variety was incubated for two hours at 100° C. in order to decarboxylate CBD-A into CBD and THC-A into THC.

In a first extraction step, 10 g of the incubated hemp and 5 g of an omega-3 fatty acid oil that contained 96.5% EPA were then mixed and left to stand for 1 h at 23° C. Filtration was subsequently performed and the filtrate formed and the filter were washed with 30 g of ethanol in order to remove the omega-3 fatty acid oil from the filter and the filter cake.

After evaporation of the ethanol, the weight (total g) of cannabinoids in the extraction product formed was determined and the concentration thereof in the extraction product formed was measured.

The extraction product was used as extractant again in a second extraction step. In the second extraction step, 5 g of the extraction product obtained after the first extraction and composed of omega-3 fatty acid oil enriched with cannabinoids was once again added to 10 g of the incubated hemp and left to stand for 1 h at 23° C. Filtration was subsequently performed once again and the filtrate formed and the filter were washed with 30 g of ethanol in order to remove the omega-3 fatty acid oil from the filter and the filter cake.

After evaporation of the ethanol, the weight (total g) of cannabinoids in the extraction product formed was determined and the concentration thereof in the extraction product formed was measured.

The extraction product that had been formed after the second extraction step was used as extractant once again in a third extraction step. In the third extraction step, 5 g of the extraction product obtained after the second extraction and composed of omega-3 fatty acid oil enriched with cannabinoids was again added to 10 g of the incubated hemp and left to stand for 1 h at 23° C. Filtration was subsequently performed once again and the filtrate formed and the filter were washed with 30 g of ethanol in order to remove the omega-3 fatty acid oil from the filter and the filter cake.

After evaporation of the ethanol, the weight (total g) of cannabinoids in the extraction product formed was determined and the concentration thereof in the extraction product formed was measured.

The measurement results are reported in the following table. They are also shown in graph form in FIGS. 1 and 2.

TABLE 2

| Measurement | CBD (% | THC (% | CBD-A (% | |
|---|---|---|---|---|
| after | by weight) | by weight) | by weight) | Total (g) |
| Extraction step 1 | 1.09 | 0.09 | 0.02 | 5.09 |
| Extraction step 2 | 2.02 | 0.169 | 0.04 | 5.61 |
| Extraction step 3 | 3.07 | 0.248 | 0.065 | 5.92 |

### 3^{RD} EXAMPLE

Hemp of the Finola variety was incubated for two hours at 100° C. in order to decarboxylate CBD-A into CBD and THC-A into THC.

Then, in nine series of experiments each with a single extraction step, 10 g of the incubated hemp and 5 g of an omega-3 fatty acid oil that contained 96.5% EPA were mixed and, as can be seen from Table 3, left to stand at different temperatures and for different durations.

Filtration was subsequently performed and the filtrate formed and the filter were washed with 30 g of ethanol in order to remove the omega-3 fatty acid oil from the filter and the filter cake.

After evaporation of the ethanol, the weight (total g) of cannabinoids in the extraction product formed was determined and the concentration thereof in the extraction product formed was measured.

TABLE 3

| % CBD | 4° C. | 30° C. | 60° C. |
|---|---|---|---|
| 30 min | 0.22 | 0.29 | 0.30 |
| 60 min | 0.24 | 0.30 | 0.33 |
| 120 min | 0.26 | 0.30 | 0.35 |

Results of this measurement are that the highest yield was achieved at the highest temperature and the longest incubation. Identical values to those at 30° C. were achieved at the highest temperature and the lowest incubation time. The lowest temperature is not suitable for the extraction due to low yield. By doubling the temperature and quadrupling the incubation time, only a 17% increase was achieved in comparison with 30° C. and 30 minute incubation.

1-20. (canceled)

21. A method for extraction, comprising using an extractant to dissolve at least one component out of a substance, in particular a plant material, wherein the extractant comprises fatty acid ethyl ester or/and fatty acid methyl ester.

22. The method according to claim 21, wherein the extractant comprises or is fatty acid ethyl ester of omega-3 and/or omega-6 fatty acid(s).

23. The method according to claim 22, wherein the extractant is EPA, DHA, DPA, ETA, 21:5n3 and/or SDA.

24. The method according to claim 21, wherein the extractant comprises a further organic solvent in addition to the fatty acid ethyl ester or/and the fatty acid methyl ester.

25. The method according to claim 24, wherein the further organic solvent is an alcohol, preferably ethanol, methanol, n-butanol and/or isopropanol.

26. The method according to claim 25, wherein the alcohol is ethanol, methanol, n-butanol and/or isopropanol.

27. The method according to claim 25, wherein the extractant is a substance mixture.

28. The method according to claim 21, wherein the at least one component is an extraction material bound in the fatty acid ethyl ester and/or fatty acid methyl ester during the extraction.

29. The method according to claim 21, wherein the extractant comprises at least 90 mg/g of fatty acid ethyl ester or/and at least 165 mg/g of fatty acid methyl ester.

30. The method according to claim 29, wherein the extractant comprises at least at least 120 mg/g of fatty acid ethyl ester or/and at least 190 mg/g of fatty acid methyl ester.

31. The method according to claim 21, wherein the extractant comprises at least 500 mg/g of fatty acid ethyl ester or/and at least 500 mg/g of fatty acid methyl ester.

32. The method according to claim 31, wherein the extractant comprises at least 700 mg/g of fatty acid ethyl ester.

33. The method according to claim 32, wherein the extractant comprises at least 900 mg/g of fatty acid ethyl.

34. The method according to claim 31, wherein the extractant comprises at least 700 mg/g of fatty acid methyl ester.

35. The method according to claim 34, wherein the extractant comprises at least 900 mg/g of fatty acid methyl ester.

36. The method according to claim 21, wherein the at least one component is a cannabinoid.

37. The method according to claim 21, wherein the extraction method is a method for producing a substance mixture containing the fatty acid ethyl ester or/and fatty acid methyl ester and an extraction material extracted from a substance.

38. The method according to claim 21, wherein the fatty acid ethyl ester contained in the substance mixture or/and the fatty acid methyl ester forms the extractant.

39. The method according to claim 27, including carrying out the method so that the substance mixture comprises at least 500 mg/g of fatty acid ethyl ester or/and at least 500 mg/g of fatty acid methyl ester.

40. The method according to claim 39, including carrying out the method so that the substance mixture comprises at least 700 mg/g of fatty acid ethyl ester.

41. The method according to claim 40, including carrying out the method so that the substance mixture comprises at least 900 mg/g of fatty acid ethyl ester.

42. The method according to claim 39, including carrying out the method so that the substance mixture comprises at least 700 mg/g of fatty acid methyl ester.

43. The method according to claim 42, including carrying out the method so that the substance mixture comprises at least 900 mg/g of fatty acid methyl ester.

44. The method according to claim 27, including carrying out the extraction method so that the substance mixture contains extraction material in a content of at least at least 5% by weight.

45. A substance mixture containing an extraction material extracted from a plant material, wherein the substance mixture comprising fatty acid ethyl ester or/and fatty acid methyl ester.

46. The substance mixture according to claim 45, wherein the fatty acid ethyl ester and/or fatty acid methyl ester is an extractant by which the extraction material has been extracted, the extraction material being dissolved in the fatty acid ethyl ester and/or fatty acid methyl ester.

47. The substance mixture according to claim 45, wherein the extraction material is a cannabinoid.

48. The substance mixture according to claim 45, wherein the substance mixture contains the extraction material in a content of at least 5% by weight.

49. The substance mixture according to claim 45, wherein the substance mixture comprises substance components of the extraction material in a ratio of the substance components to one another that at least substantially corresponds to a substance component ratio of the extraction material in the plant material.

50. The substance mixture according to claim 45, wherein the fatty acid ethyl ester comprises or is omega-3 and/or omega-6 fatty acid(s).

51. The substance mixture according to claim 45, wherein the substance mixture comprises at least 500 mg/g of fatty acid ethyl ester or/and at least 500 mg/g of fatty acid methyl ester.
